SEQUENCE LISTING (7 sequences)

SEQ ID NO: 1 - protein, 6 aa, Artificial Sequence
  Description of Artificial Sequence  protease activated receptor agonist peptide
FSLLRY

SEQ ID NO: 2 - protein, 6 aa, Artificial Sequence
  Description of Artificial Sequence  protease activated receptor agonist peptide
LSIGRL

SEQ ID NO: 3 - protein, 5 aa, Artificial Sequence
  Description of Artificial Sequence  protease activated receptor agonist peptide
LIGRL

SEQ ID NO: 4 - protein, 6 aa, Artificial Sequence
  Description of Artificial Sequence  protease activated receptor agonist peptide
SLIGRL

SEQ ID NO: 5 - protein, 6 aa, Artificial Sequence
  Description of Artificial Sequence  protease activated receptor agonist peptide
SLIGKV

SEQ ID NO: 6 - protein, 6 aa, Artificial Sequence
  Description of Artificial Sequence  protease activated receptor agonist peptide
TFLLRN

SEQ ID NO: 7 - protein, 4 aa, Artificial Sequence
  Description of Artificial Sequence  protease activated receptor agonist peptide
KNRK

(19) United States
(12) Patent Application Publication
(10) Pub. No.: US 2006/0104944 A1
(43) Pub. Date: May 18, 2006

(54) Activators and inhibitors of protease activated receptor2 (PAR2) and methods of use

(76) Inventor: Shaker A. Mousa, Wynantskill, NY (US)

Correspondence Address: Michael L. Goldman;Nixon Peabody LLP, Clinton Square, P.O. Box 31051, Rochester, NY, 14603, US

(21) Appl. No.: 11/018,710

(22) Filed: Dec. 20, 2004

Related U.S. Application Data
(60) Provisional application No. 60/629,190, filed on Nov. 18, 2004.

(51) Int. Cl. A61K 38/21; A61K 38/20; A61K 38/18; A61K 38/17; A61K 31/7076; A61K 31/7072; A61K 31/522; A61K 31/33; A61K 31/17; A61K 31/175; A61K 31/513

(52) U.S. Cl. 424/85.2; 514/9; 514/12; 424/143.1; 514/263.31; 514/49; 514/45; 424/649; 514/183; 514/588; 514/323; 424/85.4; 514/251; 514/589

(57) ABSTRACT

The present invention relates to a method of treating a subject for a condition mediated by a deficiency in angiogenesis by administering to the subject a PAR2 agonist under conditions effective to promote angiogenesis and treat the conditions mediated by a deficiency in angiogenesis. A further aspect of the present invention is a method of treating a subject for condition mediated by excessive or pathological angiogenesis by administering to the subject a PAR2 antagonist or inhibitor under conditions effective to inhibit angiogenesis and treat the subject for the condition mediated by excessive or pathological angiogenesis.

This application claims benefit of U.S. Provisional Patent Application Ser. No. 60/629,190, filed Nov. 18, 2004.

### FIELD OF THE INVENTION

The present invention relates to the use of Protease Activated Receptor 2 (PAR2) agonists in treating disorder mediated by deficient angiogenesis and to the use of PAR2 antagonists to treat a variety of excessive angiogenesis-mediated disorders.

### BACKGROUND OF THE INVENTION

PARs constitute a new and growing family of G protein-coupled receptors that are activated by proteases. The discovery of PARs highlights new functions for proteases; they can no longer be considered solely as degradative enzymes but are also signaling molecules that can specifically regulate target cells through PAR activation.

Little work has been published to date on PAR-2 antagonists. In a single report, two peptides FSLLRY-NH₂ (SEQ ID NO: 1)(Hollenberg et al., Can J. Physiol. Pharmacol. 75:832 (1997)) and LSIGRL-NH₂ (SEQ ID NO: 2)(Vergnolle et al., Br. J. Pharmacol. 127:1083 (1998)), which are N-terminal reversed sequences of PAR-1 and PAR-2 agonist peptide sequences, respectively, were shown to lack agonistic activity at their respective receptors (Al-Ani et al., J Pharmacol Exp Thera. 300:702 (2002)). These two peptides were shown to antagonize trypsin (2 nM) induced responses in KNRK (SEQ ID NO: 7) cell lines expressing rat or human PAR-2 with IC₅₀ values of 50-200 μM (Hollenberg et al., Can J. Physiol. Pharmacol. 75:832 (1997); Vergnolle et al., Br. J. Pharmacol. 127:1083 (1998)). FSLLRY-NH₂ (SEQ ID NO: 1) can also antagonize trypsin relaxant activity in rat aorta (Hollenberg et al., Can J. Physiol. Pharmacol. 75:832 (1997)).

A number of PAR-2 activating peptides are also provided herein and include SEQ ID NOs: 3-5 as follows: HOOC-LIGRLO-NH₂ (SEQ ID NO: 3), HOOC-SLIGRL-NH₂ (SEQ ID NO: 4), HOOC-SLIGKV-NH₂ (SEQ ID NO: 5), and HOOC-Fluoryl-LIGRLO-NH₂ (SEQ ID NO: 3).

A number of PAR-1 antagonists of the peptide-mimetic and nonpeptide classes have been disclosed since 1999. RWJ-56110 (Andrade-Gordon et al., Proc. Natl. Acad. Sci. USA 96:12257 (1999)) is reported to have IC₅₀=0.1-0.5 μM as a PAR-1 antagonist in a variety of bioassays but is inactive as a PAR-2 antagonist.

Presently, there are four identified members of the PAR family: three receptors for thrombin (PAR1, PAR3, and PAR4), and a single receptor for trypsin and mast cell tryptase (PAR2). All are heptahelical receptors that are activated by proteolytic cleavage, and several proteases have been identified that are capable of activating each receptor.

The discovery of a PAR family initiated intensive investigations into the functions of these receptors. The general approach has been to map receptor distribution at the tissue and cellular level, and to examine the biological effects PAR agonists in different systems. The use of these genetically modified animals in models of disease further supports an important role of these receptors in disease mechanisms. These studies have provided further impetus for the development of antagonists of this receptor family.

Trypsin and tryptase are the principal agonists of PAR2. Trypsin and tryptase cleave PAR2 at Arg³⁶ and Ser³⁷ to expose the tethered ligand SLIGRL (SEQ ID NO: 4)(rat and mouse PAR2), which then binds to conserved regions in extracellular loop II of the cleaved receptor. Glycosylation of PAR2 profoundly alters the ability of tryptase to activate this receptor (Compton et al., J. Biol. Chem. 275:39207 (2000)). Tryptase is a doughnut-shaped tetramer with active sites on the inner surface. The extracellular tail of PAR2 contains a glycosylation site in close proximity to the activation site. The potency with which tryptase activates PAR2 is dramatically increased by enzymatic deglycosylation of PAR2, or by expression of PAR2 in glycosylation-defective cells, such that tryptase becomes almost as potent as trypsin. Perhaps, the deglycosylated receptor is more readily accommodated by the active site of tryptase, allowing for efficient cleavage.

Certain coagulation factors can activate PAR2 in the presence of accessory proteins. For example, coagulation Factor VIIa activates PAR2 only in cells that also express tissue factor, an integral membrane protein which binds and concentrates Factor VIIa at the cell surface in the vicinity of PAR2 (Lerner et al., J. Biol. Chem. 271:13943 (1996)). Similarly, effector cell protease receptor-1 serves to bind and concentrate Factor Xa at the cell surface to facilitate activation of PAR2 (Al-Ani et al., Br. J. Pharmacol. 128:1105 (1999)). Certain membrane-bound proteases can also activate PAR2. Membrane-type serine protease 1 (MT-SP1) is a type II integral membrane protein, which is capable of signaling through PAR2 (Nystedt et al., Eur. J. Biochem. 232:84 (1995)). Since MT-SP1 and PAR2 have similar tissue distribution, it is conceivable that this protease could be a physiological PAR2 agonist.

Inflammatory agents and tissue damage up-regulate PAR2 in the vasculature. The inflammatory stimuli tumor necrosis factor-α (TNF-α), interleukin-1 (IL-1), and lipopoly-saccharide (LPS) upregulate PAR2 mRNA and immuno-reactivity by cultured HUVEC (human vascular endothelial cells) within 4-20 hours (Mirza et al., J. Clin. Invest. 97:1705 (1996)). Similarly, administration of bacterial endotoxin to intact rats 20 hours before analysis induces increased expression of PAR2 on endothelial and smooth muscle cells from the jugular vein and carotid artery (Storck et al., Thromb. Res. 84:463 (1996)). Incubation of segments of human coronary artery with IL-1α and TNF-α upregulates PAR2 mRNA levels within 12 hours (Alm et al., Thromb. Haemostasis 81:984 (1999)). Vascular injury also upregulates PAR2 expression. Injury of the rat carotid artery with a balloon catheter stimulates PAR2 expression within days, particularly by proliferating smooth muscle cells of the neointima (Belham et al., Biochem. J. 320:9 (1996)). Together, these findings suggest a role for PAR2 in inflammation and injury of the vascular system.

The expression of PAR2 on endothelial cells and vascular smooth muscle of arteries and veins suggests that PAR2 regulates vascular tone. The effects of PAR2 agonists have been extensively studied in isolated tissues suspended in organ baths to measure contraction and by measuring alterations in arterial pressure in intact animals. The predominant effect of PAR2 agonists is to induce relaxation of blood vessels and consequent hypotension. Trypsin and PAR2 activating peptides cause an endothelium-dependent relaxation of isolated preparations from rat (Bretschneider et al., Br. J. Pharmacol. 126:1735 (1999)) and rabbit aorta (Saifeddine et al., J. Pharmacol. 118:521 (1996)), human (Hollenberg et al., Can. J. Physiol. Pharmacol. 75:832 (1997)). The mechanism of arterial relaxation involves Ca²⁺ mobilization and subsequent activation of nitric oxide synthetase in endothelial cells, followed by release of nitric oxide, which relaxes vascular smooth muscle. Indeed, PAR2 agonists stimulate release of nitric oxide from rat aortic rings. Moreover, an analogue of the PAR2 activating peptide, tc-LIGRLO (SEQ ID NO: 3), which is known to be a very selective PAR2 agonist, does not cause contraction, while it was equally active with SLIGRL (SEQ ID NO: 4) in the relaxation assay (Saifeddine et al., J. Pharmacol. 118:521 (1996)).

PAR2 agonists also have major effects on the vasculature of intact animals. Intravenous injection of PAR2 agonists causes an NO-dependent hypotension in rats and mice (Kawabata et al., Br. J. Pharmacol. 125:419 (1998); Vergnolle et al., Br. J. Pharmacol. 127:1083 (1999)). The specificity of PAR2 activation-induced vascular response has been confirmed by experiments on PAR2 deficient mice (Saifeddine et al., Br. J. Pharmacol. 125:1445 (1998)).

Agonists of PAR2 in the Vascular System

Trypsin is the most potent agonist of PAR2, and trypsinogen is expressed by endothelial cells. Whether trypsins from endothelial cells can regulate the vasculature through PAR2 remains to be determined. Mast cell proteases, such as tryptase, may also activate PAR2 on endothelial or vascular smooth muscle cells. Indeed, mast cell numbers increase at sites of vascular injury (Cheung et al., Can. J. Physiol. Pharmacol. 76:16 (1998)). Alternatively, certain coagulation factors could trigger PAR2. Factor VIIa activates PAR2 in cells that also express tissue factor, which binds and concentrates Factor VIIa at the cell surface in the vicinity of PAR2 (Lerner et al., J. Biol. Chem. 271:13943 (1996)). Factor Xa can also activate endothelial cells by a novel cascade that involves binding of Factor Xa to effector cell protease receptor-1 (EPR-1) at the cell surface. This serves to concentrate Factor Xa and facilitate cleavage and activation of PAR2 (Al-Ani et al., Br. J. Pharmacol. 128:1105 (1999); Cicala et al., Circulation 99:2590 (1999)). Factor Xa induces mitogenesis of human coronary artery smooth muscle cells by a signaling pathway triggered by PAR2 (Vergnolle, N. J. Immunol. 163 :5064 (1999)).

Pro-Inflammatory Effects of PAR2

Numerous studies suggest that PAR2 makes an important contribution to the development of inflammation. PAR2 activation leads to increased vascular permeability, vascular relaxation (Kawabata et al., J. Pharmacol. Exp. Ther. 288:358 (1999)), and granulocyte infiltration (Mule et al., Br. J. Pharmacol. 136:367 (2002)). The role of PAR2 in the induction of inflammatory responses has been confirmed using PAR2 deficient mice. PAR2 activating peptide reduces leukocyte rolling velocity, increases leukocyte rolling flux, and increases leukocyte adhesion in wild type mice (Cocks et al., Pulm. Pharmacol. Ther. 14:183 (2001)). These inflammatory responses, which can be induced by surgical exposure of tissues, are delayed in PAR2 deficient mice, suggesting that PAR2 is an important receptor in inflammation and appears necessary for some of the earliest inflammatory responses in vivo.

PAR2 is a prominent member of this receptor family which plays an important role in diverse physiological and pathophysiological processes in many organ systems, including the cardiovascular system, the pulmonary system, the gastrointestinal tract, and the skin. Experimental activation of PAR2 in these systems by using proteases and activating peptides indicates that this receptor participates in many important processes relating to tissue responses to injury when proteases are generated or secreted. Thus, PAR2 participates in the processes of inflammation, pain sensation, and repair.

Despite considerable progress in understanding of the function of PAR2, there are many unanswered questions. In many tissues, the proteases that activate PAR2 are unknown. Although trypsin, tryptase, and certain coagulation factors are possible agonists, the enzymes that principally activate the receptor in different tissues remain to be identified. It will be crucial to evaluate animals lacking specific proteases or receptors or animals over-expressing PAR2 to define unequivocally the role of this receptor and its agonists in disease processes.

Angiogenesis

Angiogenesis is the development of new blood vessels from preexisting blood vessels (Mousa, In: Angiogenesis Inhibitors and Stimulators: Potential Therapeutic Implications, Landes Bioscience Inc., Georgetown, Tex.; Chapter 1 (2000)). Physiologically, angiogenesis ensures proper development of mature organisms, prepares the womb for egg implantation, and plays a key role in wound healing. On the other hand, angiogenesis supports the pathological conditions associated with a number of disease states such as cancer, inflammation, and ocular diseases.

Angiogenesis or “neovascularization” is a multi-step process controlled by the balance of pro- and anti-angiogenic factors. The latter stages of this process involve proliferation and the organization of endothelial cells (EC) into tube-like structures. Growth factors such as FGF2 and VEGF are thought to be key players in promoting endothelial cell growth and differentiation. The endothelial cell is the pivotal component of the angiogenic process and responds to many cytokines through its cell surface receptors and intracellular signaling mechanisms. Endothelial cells in culture are capable of forming tube-like structures that possess lumens.

It has been proposed that inhibition of angiogenesis would be a useful therapy for restricting tumor growth. Inhibition of angiogenesis can be achieved by inhibiting endothelial cell response to angiogenic stimuli, as suggested by Folkman et al., Cancer Biology 3:89-96 (1992), where examples of endothelial cell response inhibitors (e.g., angiostatic steroids), fungal derived products (e.g., fumagilin), platelet factor 4, thrombospondin, alpha-interferon, vitamin D analogs, and D-penicillamine, are described. For additional proposed inhibitors of angiogenesis, see Blood et al., Bioch. Biophys. Acta 1032:89-118 (1990); Moses et al., Science 248:1408-1410 (1990); and U.S. Pat. Nos. 5,092,885, 5,112,946, 5,192,744, and 5,202,352.

Control of angiogenesis is a complex process involving local release of vascular growth factors (Carmeliet, Ann NY Acad Sci 902:249-260 (2000)), extracellular matrix, adhesion molecules, and metabolic factors (Tomanek et al., Anat Rec 261:126-135 (2000)). Mechanical forces within blood vessels may also play a role (Hudlicka, Molec Cell Biochem 147:57-68 (1995)). The principal classes of endogenous growth factors implicated in new blood vessel growth are the fibroblast growth factor (FGF) family and vascular endothelial growth factor (VEGF)(Pages, Ann NY Acad Sci 902:187-200 (2000)). The mitogen-activated protein kinase (MAPK; ERK1/2) signal transduction cascade is involved both in VEGF gene expression and in control of proliferation of vascular endothelial cells.

The availability of a chick chorioallantoic membrane (CAM) assay for angiogenesis has provided a model in which to quantitate angiogenesis (Auerbach et al., Dev Biol. 41:391-394 (1974); Powell et al., J. Cellular Biochemistry 80:104-114 (2000); Dupont et al., Clin Exp Metastasis 19:145-153 (2002); Kim et al., Am. J Pathol 156:1345-1362 (2000); Colman et al., Blood 95(2):543-550 (2000); Colman et al., J Thrombosis Haemostasis 1(l):164-173 (2003); Ali, et al., Cancer Research 60:7094-7098 (2000); Van Waes et al., Int. J Oncology 16:1189-1195 (2000); Luna et al., Lab Investigation 75:563-573 (1996)).

### SUMMARY OF THE INVENTION

One aspect of the present invention relates to a method of treating a subject for a condition mediated by a deficiency in angiogenesis. This method involves administering to the subject a par2 agonist or activator under conditions effective to promote angiogenesis and treat the conditions mediated by a deficiency in angiogenesis. Such conditions include the following: impaired wound healing, erectile dysfunction (impotence), acute myocardial infarction (heart attack), stroke or transient ischemic attack, peripheral artery diseases, stomach ulcer or foot ulceration, infertility, and other vascular disorders.

Another aspect of the present invention relates to a method of treating a subject for a condition mediated by excessive or pathological angiogenesis. This method involves administering to the subject a PAR2 antagonist or inhibitor under conditions effective to inhibit angiogenesis and treat the subject for the condition mediated by excessive or pathological angiogenesis. Such conditions include the following: solid tumor growth, tumor metastasis, multiple myeloma, lymphoma, ocular angiogenesis-mediated disorders (diabetic retinopathy, macular degeneration, and other ocular angiogenesis disorders), and angiogenesis-mediated inflammatory disorders.

The invention is based, in part, on the discovery that PAR2 agonists, and their polymeric forms have pro-angiogenesis properties. Accordingly, these PAR2 agonists, and polymeric forms (i.e., pro-angiogenesis agents) can be used to treat a variety of deficient angiogenesis-mediated disorders. Similarly, the invention is also based on the discovery that PAR2 antagonists inhibit the pro-angiogenesis effect of PAR2 activation, other pro-angiogenesis factors and can also be used to treat a variety of excessive angiogenesis-mediated disorders.

PAR2 activating peptides (SEQ ID NOs: 3-5), trypsin, tryptase, coagulation Factor VIIa or Factor Xa-induced activation of PAR2 and promoted angiogenesis in comparable way to that observed with VEGF or FGF2.

The present invention provides a novel strategy in the prevention and treatment of various vascular-related disorders ranging from inflammatory, cancer, and ocular-mediated disorders.

The present invention is directed to the observation that PAR2 agonist promoted angiogenesis, while PAR2 blockade provided potent anti-angiogenesis properties against FGF2, PAR2 agonist, trypsin, other growth factors, or Tissue Factor/Factor VIIa/Xa-mediated angiogenesis.

### BRIEF DESCRIPTION OF THE DRAWINGS

FIG. 1 shows the effects of PAR2 agonists on angiogenesis quantitated in the chick CAM assay. Control samples were exposed to PBS and additional groups to PAR2 activating peptides (SEQ ID NOs: 3-5) for 3 days. PAR2 agonists caused increased blood vessel branching in these representative images from 3 experiments. PAR2 agonists increased blood vessel formation that is comparable to basic fibroblast growth factor or vascular endothelial cell growth factor.

FIG. 2 shows that a PAR2 blocking antibody inhibits stimulation of angiogenesis by PAR2 activating peptides and by other pro-angiogenesis growth factors. A 2-fold increase in blood vessel branch formation is seen in a representative CAM preparation exposed to 1-10 ng of PAR2 agonists for 3 days. This effect is inhibited by PAR2 monoclonal antibody and by Factor VII/VIIa antibody.

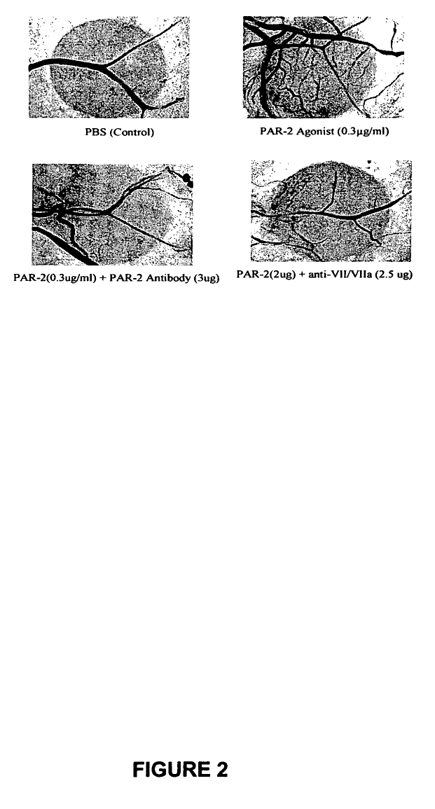

FIG. 3 shows the effect of ανβ3 integrin antagonist (XT199), thrombin inhibitor (Hirudin), MAP kinase inhibitor (PD98059) on PAR2 activating peptide-induced angiogenesis in the CAM model. PAR2-induced angiogenesis is blocked by the avb3 antagonists, XT199 or MAP kinase (PD98059) but not by the thrombin inhibitor hirudin.

FIG. 4 shows an increase in sprouting by PAR2 agonist administration.

FIG. 5 shows in vitro wound healing promotion by a PAR2 agonist.

### DETAILED DESCRIPTION OF THE INVENTION

One aspect of the present invention relates to a method of treating a subject for a condition mediated by a deficiency in angiogenesis. This method involves administering to the subject a PAR2 agonist or activator under conditions effective to promote angiogenesis and treat the conditions mediated by a deficiency in angiogenesis. Suitable PAR2 agonists or activators include the following: activating peptides (SEQ ID NOs: 3-5), trypsin, tryptase, coagulation Factor VIIa and/or Factor Xa.

Another aspect of the present invention relates to a method of treating a subject for a condition mediated by excessive or pathological angiogenesis. This method involves administering to the subject a PAR2 antagonist or inhibitor under conditions effective to inhibit angiogenesis and treat the subject for the condition mediated by excessive or pathological angiogenesis. Suitable PAR2 antagonists or inhibitors include monoclonal antibodies against PAR2, peptides, cyclic peptides, peptidomimetics, and inhibitors for trypsin, tryptase, anti-Factor Xa, anti-Factor VIIa, or combinations thereof.

The features and other details of the invention will now be more particularly described with references to the accompanying drawings. For convenience, certain terms used in the present invention are collected below. Unless otherwise defined, all technical and scientific terms used herein have the same meaning as commonly understood by one of ordinary skill in the art to which this invention pertains.

As used herein, the term “angiogenic agent” includes any compound or substance that promotes or encourages angiogenesis, whether alone or in combination with another substance. Examples include, but are not limited to PAR2 agonists (SEQ ID NOs: 3-5) in linear, cyclic, peptidomimetic, non-peptide, or polymeric analogs. In contrast, the terms “anti-angiogenesis agent” or anti-angiogenic agent” refer to any compound or substance that inhibits or discourages angiogenesis, whether alone or in combination with another substance. Examples include, but are not limited to, PAR2 antagonists that include monoclonal antibody, fragment, peptide such peptide sequence E or F, or non-peptide.

As used herein, the term “myocardial ischemia” is defined as an insufficient blood supply to the heart muscle caused by a decreased capacity of the heart vessels. As used herein, the term “coronary disease” is defined as diseases/disorders of cardiac function due to an imbalance between myocardial function and the capacity of coronary vessels to supply sufficient blood flow for normal function. Specific coronary diseases/disorders associated with coronary disease which can be treated with the compositions and methods described herein include myocardial ischemia, angina pectoris, coronary aneurysm, coronary thrombosis, coronary vasospasm, coronary artery disease, coronary heart disease, coronary occlusion and coronary stenosis.

As used herein the term “occlusive peripheral vascular disease” (also known as peripheral arterial occlusive disorder) is a vascular disorder-involving blockage in the carotid or femoral arteries, including the iliac artery. Blockage in the femoral arteries causes pain and restricted movement. A specific disorder associated with occlusive peripheral vascular disease is diabetic foot, which affects diabetic patients, often resulting in amputation of the foot.

As used herein the terms “regeneration of blood vessels,” “angiogenesis,” “revascularization,” and “increased collateral circulation” (or words to that effect) are considered synonymous.

The term “pharmaceutically acceptable” when referring to a natural or synthetic substance means that the substance has an acceptable toxic effect in view of its much greater beneficial effect, while the related the term, “physiologically acceptable,” means the substance has relatively low toxicity.

The term, “co-administered” means two or more drugs are given to a patient at approximately the same time or in close sequence so that their effects run approximately concurrently or substantially overlap. This term includes sequential as well as simultaneous drug administration.

“Pharmaceutically acceptable salts” refers to pharmaceutically acceptable salts of PAR2 agonists, polymeric forms, and derivatives, which salts are derived from a variety of organic and inorganic counter ions well known in the art and include, by way of example only, sodium, potassium, calcium, magnesium, ammonium, tetra-alkyl ammonium, and the like. When the molecule contains a basic functionality, salts of organic or inorganic acids, such as hydrochloride, hydro-bromide, tartrate, mesylate, acetate, maleate, oxalate and the like, can be used as the pharmaceutically acceptable salt.

“Subject” includes living organisms such as humans, monkeys, cows, sheep, horses, pigs, cattle, goats, dogs, cats, mice, rats, cultured cells, and transgenic species thereof. In a preferred embodiment, the subject is a human. The methods of administration in accordance with the present invention can be carried out using known procedures, at dosages and for periods of time effective to treat the condition in the subject. An effective amount of the therapeutic compound necessary to achieve a therapeutic effect may vary according to factors such as the age, sex, and weight of the subject, and the ability of the therapeutic compound to treat the foreign agents in the subject. Dosage regimens can be adjusted to provide the optimum therapeutic response. For example, several divided doses may be administered daily or the dose may be proportionally reduced as indicated by the exigencies of the therapeutic situation.

“Administering” includes routes of administration which allow the compositions of the invention to perform their intended function, e.g., promoting or inhibiting angiogenesis. A variety of routes of administration are possible, including, but not necessarily limited to, parenteral (e.g., intravenous, intra-arterial, intramuscular, subcutaneous injection), oral (e.g., dietary), topical, nasal, rectal, or via slow releasing micro-carriers depending on the disease or condition to be treated. Oral, parenteral, and intravenous administration are preferred modes of administration. Formulation of the compound to be administered will vary according to the route of administration selected (e.g., solution, emulsion, gels, aerosols, capsule). An appropriate composition comprising the compound to be administered can be prepared in a physiologically acceptable vehicle or carrier and optional adjuvant and preservatives. For solutions or emulsions, suitable carriers include, for example, aqueous or alcoholic/aqueous solutions, emulsions or suspensions, including saline and buffered media, sterile water, creams, ointments, lotions, oils, pastes and solid carriers.

Parenteral vehicles can include sodium chloride solution, Ringer's dextrose, dextrose, and sodium chloride, lactated Ringer's or fixed oils. Intravenous vehicles can include various additives, preservatives, or fluid, nutrient or electrolyte replenishers (See generally, Remington's Pharmaceutical Science, 16th Edition, Mack, Ed. (1980), which is hereby incorporated by reference in its entirety). Formulations suitable for parenteral administration conveniently include sterile aqueous preparation of the active compound, which is preferably isotonic with the blood of the recipient. Thus, such formulations may conveniently contain distilled water, 5% dextrose in distilled water or saline. Useful formulations also include concentrated solutions or solids containing the compound of formula (I), which upon dilution with an appropriate solvent give a solution suitable for parental administration above.

For enteral administration, a compound can be incorporated into an inert carrier in discrete units such as capsules, cachets, tablets, or lozenges, each containing a predetermined amount of the active compound; as a powder or granules; or a suspension or solution in an aqueous liquid or non-aqueous liquid, e.g., a syrup, an elixir, an emulsion, or a draught. Suitable carriers may be starches or-sugars and include lubricants, flavorings, binders, and other materials of the same nature.

A tablet may be made by compression or molding, optionally with one or more accessory ingredients. Compressed tablets may be prepared by compressing in a suitable machine the active compound in a free-flowing form, e.g., a powder or granules, optionally mixed with accessory ingredients, e.g., binders, lubricants, inert diluents, surface active or dispersing agents. Molded tablets may be made by molding in a suitable machine, a mixture of the powdered active compound with any suitable carrier.

A syrup or suspension may be made by adding the active compound to a concentrated, aqueous solution of a sugar, e.g., sucrose, to which may also be added any accessory ingredients. Such accessory ingredients may include flavoring, an agent to retard crystallization of the sugar or an agent to increase the solubility of any other ingredient, e.g., as a polyhydric alcohol, for example, glycerol or sorbitol.

Formulations for rectal administration may be presented as a suppository with a conventional carrier, e.g., cocoa butter or Witepsol S55 (trademark of Dynamite Nobel Chemical, Germany), for a suppository base.

Alternatively, the compound may be administered in liposomes or microspheres (or microparticles). Methods for preparing liposomes and microspheres for administration to a patient are well known to those of skill in the art. U.S. Pat. No. 4,789,734, which is hereby incorporated by reference in its entirety, describes methods for encapsulating biological materials in liposomes. Essentially, the material is dissolved in an aqueous solution, the appropriate phospholipids and lipids added, along with surfactants if required, and the material dialyzed or sonicated, as necessary. A review of known methods is provided by G. Gregoriadis, Chapter 14, “Liposomes,” Drug Carriers in Biology and Medicine, pp. 287-341 (Academic Press, 1979), which is hereby incorporated by reference in its entirety.

Microspheres formed of polymers or proteins are well known to those skilled in the art and can be tailored for passage through the gastrointestinal tract directly into the blood stream. Alternatively, the compound can be incorporated and the microspheres or composite of microspheres, implanted for slow release over a period of time ranging from days to months. See, for example, U.S. Pat. Nos. 4,906,474, 4,925,673 and 3,625,214, and Jein, TIPS 19:155-157 (1998), which are hereby incorporated by reference in their entirety.

In one embodiment, the PAR2 agonist or its polymeric forms, and adenosine derivatives can be formulated into a liposome or microparticle, which is suitably sized to lodge in capillary beds following intravenous administration. When the liposome or microparticle is lodged in the capillary beds surrounding ischemic tissue, the agents can be administered locally to the site at which they can be most effective. Suitable liposomes for targeting ischemic tissue are generally less than about 200 nanometers and are also typically unilamellar vesicles, as disclosed, for example, in U.S. Pat. No. 5,593,688 to Baldeschweiler, which is hereby incorporated by reference in its entirety.

Preferred microparticles are those prepared from biodegradable polymers, such as polyglycolide, polylactide and copolymers thereof. Those of skill in the art can readily determine an appropriate carrier system depending on various factors, including the desired rate of drug release and the desired dosage.

In one embodiment, the agonist of the present invention is administered via catheter directly to the inside of blood vessels. The administration can occur, for example, through holes in the catheter. In that embodiment, where the active compounds have a relatively long half life (on the order of 1 day to a week or more), the formulations can be included in biodegradable polymeric hydrogels, such as those disclosed in U.S. Pat. No. 5,410,016 to Hubbell et al., which is hereby incorporated by reference in its entirety. These polymeric hydrogels can be delivered to the inside of a tissue lumen and the active compounds released over time as the polymer degrades. If desirable, the polymeric hydrogels can have microparticles or liposomes which include the active compound dispersed therein, providing another mechanism for the controlled release of the active compounds.

“Effective amount” includes those amounts of pro-angiogenic or anti-angiogenic compounds which allow it to perform its intended function, e.g., promoting or inhibiting angiogenesis in angiogenesis-related disorders as described herein. The effective amount will depend upon a number of factors, including biological activity, age, body weight, sex, general health, severity of the condition to be treated, as well as appropriate pharmacokinetic properties. For example, dosages of the active substance may be from about 0.01 mg/kg/day to about 500 mg/kg/day, advantageously from about 0.1 mg/kg/day to about 100 mg/kg/day. A therapeutically effective amount of the active substance can be administered by an appropriate route in a single dose or multiple doses. Further, the dosages of the active substance can be proportionally increased or decreased as indicated by the exigencies of the therapeutic or prophylactic situation.

“Pharmaceutically acceptable carrier” includes any and all solvents, dispersion media, coatings, antibacterial and antifungal agents, isotonic and absorption delaying agents, and the like which are compatible with the activity of the compound and are physiologically acceptable to the subject. An example of a pharmaceutically acceptable carrier is buffered normal saline (0.15 M NaCl). The use of such media and agents for pharmaceutically active substances is well known in the art. Except insofar as any conventional media or agent is incompatible with the therapeutic compound, use thereof in the compositions suitable for pharmaceutical administration is contemplated. Supplementary active compounds can also be incorporated into the compositions.

“Additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be used in conjunction with the present invention are known in the art and described, e.g., in Remington's Pharmaceutical Sciences, which is hereby incorporated by reference in its entirety.

Accordingly, one aspect the present invention features methods for treating a condition amenable to treatment by promoting angiogenesis by administering to a subject in need thereof an amount of a polymeric form of PAR2 agonist peptides, or an analog thereof, effective for promoting angiogenesis. Examples of such conditions amenable to treatment by promoting angiogenesis are provided herein and can include occlusive vascular disease, coronary disease, erectile dysfunction, myocardial infarction, ischemia, stroke, peripheral artery vascular disorders, and wounds. Venous thromboembolic disorders (deep vein thrombosis, sickle cell diseases, and pulmonary embolism), arterial thromboembolic disorders (coronary artery diseases, cerebrovascular disorders, and peripheral artery diseases), myocardial infarction, ischemia, erectile dysfunction, and stroke can all be treated in accordance with the present invention.

Examples of PAR2 agonist are also provided herein and can include linear, cyclic, peptidomimetic or non-peptide analogs of the following peptide sequences:

| | |
|---|---|
| HOOC-L-I-G-R-L-O-NH₂, | (SEQ ID NO: 3) |
| HOOC-S-L-I-G-R-L-NH₂, | (SEQ ID NO: 4) |
| HOOC-S-L-I-G-K-V-NH₂, | (SEQ ID NO: 5) |
| 2-Fluroyl-L-I-G-R-L-O-NH₂ | (SEQ ID NO: 3) |

These PAR2 activating peptides or agonists can be in linear, cyclic, or conjugated to polyvinyl alcohol, acrylic acid ethylene co-polymer, poly-lactic acid, or polyethylene glycol on the carboxylic acid terminal. The conjugation is via covalent or non-covalent bonds depending on the polymer used.

In yet a further aspect, the present invention provides compositions (i.e., pro-angiogenesis agents) that include PAR agonists (activating peptides, A-D), and analogs conjugated to a polymer. The conjugation can be through a covalent or non-covalent bond, depending on the polymer. A covalent bond can occur through an ester or anhydride linkage, for example. In one embodiment, the polymer can include, but is not limited to, polyvinyl alcohol, acrylic acid ethylene co-polymer, polylactic acid, or polyethylene glycols.

Polymer conjugations are used to improve drug viability. While many old and new therapeutics are well-tolerated, many compounds need advanced drug discovery technologies to decrease toxicity, increase circulatory time, or modify biodistribution. One strategy for improving drug viability is the utilization of water-soluble polymers. Various water-soluble polymers have been shown to modify biodistribution, improve the mode of cellular uptake, change the permeability through physiological barriers, and modify the rate of clearance through the body. To achieve either a targeting or sustained-release effect, water-soluble polymers have been synthesized that contain drug moieties as terminal groups, as part of the backbone, or as pendent groups on the polymer chain.

Representative compositions of the present invention include PAR2 agonists (SEQ ID NOs: 3-5), analogs thereof conjugated to polymers. Conjugation with polymers can be either through covalent or non-covalent linkages. In preferred embodiments, the polymer conjugation can occur through an ester linkage or an anhydride linkage. In this preparation, commercially available polyvinyl alcohol (or related co-polymers) can be esterified by treatment with the acid chloride of PAR2 agonist, including the acid chloride form. The hydrochloride salt is neutralized by the addition of triethylamine to afford triethylamine hydrochloride which can be washed away with water upon precipitation of the PAR2 agonist ester polymer form for different analogs. The ester linkage to the polymer may undergo hydrolysis in vivo to release the active pro-angiogenesis PAR2 agonist.

A polymer conjugation through an anhydride linkage using acrylic acid ethylene co-polymer is similar to the previous polymer covalent conjugation; however, this time it is through an anhydride linkage that is derived from reaction of an acrylic acid co-polymer. This anhydride linkage is also susceptible to hydrolysis in vivo to release the PAR2 agonist. Neutralization of the hydrochloric acid is accomplished by treatment with triethylamine and subsequent washing of the precipitated polyanhydride polymer with water removes the triethylamine hydrochloride byproduct. This reaction will lead to the formation of PAR2 agonist—acrylic acid co-polymer+triethylamine. Upon in vivo hydrolysis, the PAR2 agonist will be released over time with acrylic acid ethylene Co-polymer.

Another representative polymer conjugate includes a PAR2 agonist conjugated to polyethylene glycol (PEG). Attachment of PEG to various drugs, proteins, and liposome has been shown to improve residence time and decrease toxicity. PEG can be coupled to active agents through the hydroxyl groups at the ends of the chains and via other chemical methods. PEG itself, however, is limited to two active agents per molecule. In a different approach, copolymers of PEG and amino acids were explored as novel biomaterials which would retain the biocompatibility properties of PEG, but which would have the added advantage of numerous attachment points per molecule and which could be synthetically designed to suit a variety of applications.

Another representative polymer conjugate includes PAR2 agonist non-covalently conjugated with polymers. A preferred non-covalent conjugation technique involves entrapment of PAR2 agonist in a polylactic acid polymer. Polylactic acid polyester polymers (PLA) undergo hydrolysis in vivo to the lactic acid monomer, and this has been exploited as a vehicle for drug delivery systems in humans. Unlike the prior two covalent methods where the PAR2 agonist is linked by a chemical bond to the polymer, this would be a non-covalent method that would encapsulate the PAR2 agonist peptide or analogs in PLA polymer beads. This reaction will lead to the formation of PAR2 agonist containing PLA beads in water. Filtration and washing will result in the formation of PAR2 agonist containing PLA beads, which upon in vivo hydrolysis will lead to the generation of controlled levels of PAR2 agonist plus lactic acid.

The composition of the present invention may include an effective amount of polymeric forms of PAR2 agonist peptides, analogs and derivatives and an effective amount of an adenosine and/or nitric oxide donor. The compositions can be in the form of a sterile, injectable, pharmaceutical formulation that includes an angiogenically effective amount of PAR2 agonist and adenosine derivatives in a physiologically and pharmaceutically acceptable carrier, optionally with one or more excipients.

In another embodiment, PAR2 agonists, or polymeric forms thereof can be encapsulated or incorporated in a microparticle, liposome, or polymer. The polymer can include, for example, polyglycolide, polylactide, or co-polymers thereof. The liposome or microparticle has a size of about less than 200 nanometers and can be administered via one or more parenteral routes or another mode of administration. In another embodiment, the liposome or microparticle can be lodged in capillary beds surrounding ischemic tissue, or applied to the inside of a blood vessel via a catheter. PAR2 agonist peptides, analogs, polymeric forms and derivatives can be targeted to ischemic tissue by covalent linkage with a suitable antibody.

PAR2 agonists or polymeric forms thereof according to the present invention can also be co-administered with one or more biologically active substances that can include, for example, growth factors, vasodilators, anti-coagulants, anti-virals, anti-bacterials, anti-inflammatories, immuno-suppressants, analgesics, vascularizing agents, or cell adhesion molecules, or combinations thereof. In one embodiment, the PAR2 agonists or polymeric form is administered as a bolus injection prior to or post-administering one or more biologically active substance.

Growth factors can include, for example, basic fibroblast growth factor, vascular endothelial growth factor, epithelial growth factor, nerve growth factor, platelet-derived growth factor, and vascular permeability factor. Vasodilators can include, for example, adenosine, adenosine derivatives, or combinations thereof. Anticoagulants include, but are not limited to, heparin, heparin derivatives, anti-Factor Xa, anti-thrombin, aspirin, clopidgrel, or combinations thereof.

In another aspect of the present invention, methods are provided for promoting angiogenesis along or around a medical device by coating the device with a PAR2 agonist or polymeric form thereof according to the present invention prior to inserting the device into a patient. The coating step can further include coating the device with one or more biologically active substance, such as, but not limited to, a growth factor, a vasodilator, an anti-coagulant, or combinations thereof. Examples of medical devices that can be coated with PAR2 agonists or polymeric forms according to the present invention include stents, catheters, cannulas, or electrodes.

In a further aspect, the present invention provides methods for treating a condition amenable to treatment by inhibiting angiogenesis by administering to a subject in need thereof an amount of PAR2 antagonists as an anti-angiogenesis agent (effective in inhibiting angiogenesis). PAR antagonists can include, for example monoclonal antibody (single or double chain), fragment or epitope that is capable of blocking PAR2. PAR2 antagonists could be analogs of the PAR2 activating peptide series listed above.

Examples of the conditions amenable to treatment by inhibiting angiogenesis include, but are not limited to, tumors and cancer associated disorders, retinal tumor growth, benign tumors (e.g., hemangiomas, acoustic neuromas, neurofibromas, trachomas, and pyogenic granulomas), solid tumors, blood borne tumors (e.g., leukemias, angiofibromas, and kaposi sarcoma), tumor metastases, and other cancers which require neovascularization to support tumor growth), ocular neovascular-disorders (e.g., diabetic retinopathy, macular degeneration, retinopathy of prematurity, neovascular glaucoma, corneal graft rejection, and other ocular angiogenesis-mediated disorders), inflammatory disorders (e.g., immune and non-immune inflammation, rheumatoid arthritis, chronic articular rheumatism, inflammatory bowel diseases, psoriasis, and other chronic inflammatory disorders), endometriosis, other disorders associated with inappropriate or inopportune invasion of vessels (e.g., retrolental fibroplasia, rubeosis, and capillary proliferation in atherosclerotic plaques and osteoporosis), Osler-Webber Syndrome, myocardial angiogenesis, plaque neovascularization, telangiectasia, hemophiliac joints, and wound granulation. Other diseases in which angiogenesis plays a role in the maintenance or progression of the pathological state are known to those skilled in the art and are similarly intended to be included within the meaning of the term “angiogenesis-mediated” used herein.

In one embodiment, the PAR2 antagonist or inhibitor or composition is used in conjunction with other angiogenesis inhibitors. Angiogenic inhibitors are known in the art and can be prepared by known methods. For example, angiogenic inhibitors include integrin inhibitory compounds such as, αν integrin inhibitory antibodies, cell adhesion proteins, or functional fragments thereof which contain a cell adhesion binding sequence. Additional angiogenic inhibitors include, for example, angiostatin (see, e.g., U.S. Pat. No. 5,639,725, which is hereby incorporated by reference in its entirety), functional fragments of angiostatin, endostatin (see, e.g., WO 97/15666, which is hereby incorporated by reference in its entirety), fibroblast growth factor (FGF) inhibitors, FGF receptor inhibitors, VEGF inhibitors (VEGF antibodies, VEGF trap, VEGF receptor blockers, and other mechanisms of VEGF inhibition), thrombospondin, platelet factor 4, interferon, interleukin 12, thalidomide, and compounds involved in other mechanisms for the inhibition of angiogenesis. For a description of angiogenic inhibitors and targets set forth above, see, for example, Chen et al., Cancer Rres. 55:4230-4233 (1995), Good et al., Proc. Natl. Acad. Sci. USA 87:6629-6628 (1990), O'Reilly et al., Cell 79:315-328 (1994), Parangi et al., Proc. Natl. Acad. Sci. USA 93:2002-2007 (1996), Rastinejad et al., Cell 56:345-355 (1989), Gupta et al., Proc. Natl. Acad. Sci. USA 92:7799-7803 (1995), Maione et al., Science 247:77-79 (1990), Angiolillo et al., J. Exp. Med. 182:155-162 (1995), Strieter et al., Biochem. Biophys. Res. Comm. 210:51-57 (1995); Voest et al., J. Natl. Cancer Inst. 87:581-586 (1995), Cao et al., J. Exp. Med. 182:2069-2077 (1995), Clapp et al., Endocrinology 133:1292-1299 (1993), Blood et al., Bioch. Biophys Acta. 1032:89-118 (1990), Moses et al., Science 248:1408-1410 (1990), Ingber et al., Lat Invest. 59:44-51 (1988), and U.S. Pat. Nos. 5,092,885 and 5,112,946, which are hereby incorporated by reference in their entirety.

In another embodiment, the PAR2 antagonists or inhibitor or composition is used in conjunction with other therapies, such as standard anti-inflammatory therapies, standard ocular therapies, standard dermal therapies, radiotherapy, tumor surgery, and conventional chemotherapy directed against solid tumors and for the control of establishment of metastases. The administration of the angiogenesis inhibitor is typically conducted during or after chemotherapy at time where the tumor tissue should respond to toxic assault by inducing angiogenesis to recover by the provision of a blood supply and nutrients to the tumor tissue. Additionally, it is preferred to administer such angiogenesis inhibitors after surgery where solid tumors have been removed as a prophylaxis against metastasis. Cytotoxic or chemotherapeutic agents are those known in the art such as aziridine thiotepa, alkyl sulfonate, nitrosoureas, platinum complexes, alkylators, folate analogs, purine analogs, adenosine analogs, pyrimidine analogs, substituted urea, anti-tumor antibiotics, microtubulle agents, and asprignase.

In one embodiment, the anti-angiogenesis agent is administered by a parenteral, oral, rectal, or topical mode, or combination thereof. In another embodiment, the anti-angiogenesis agent can be co-administered with one or more anti-angiogenesis therapies, radiotherapy or chemotherapeutic agents.

The pharmaceutical formulations according to the present invention can be encapsulated or incorporated in a liposome, microparticle, or polymer. The liposome or microparticle has a size of less than about 200 nanometers. Any of the pharmaceutical formulations according to the present invention can be administered via parenteral, oral, rectal, or topical means, or combinations thereof. In another embodiment, the pharmaceutical formulations can be co-administered to a subject in need thereof with one or more biologically active substances including, but not limited to, growth factors, vasodilators, anti-coagulants, or combinations thereof.

Inhibitors or antagonists of protease activated receptors (PARs 1-4) and in particular PAR2 are potent inhibitors of angiogenesis and inflammatory-mediated processes alone or in combination with other existing anti-inflammatory, anti-angiogenesis, anti-cancer, and ocular therapies for the prevention and treatment of angiogenesis-mediated disorders, cancer (e.g., primary or metastatic tumors), inflammatory, and ocular diseases (e.g., diabetic retinopathy). It is known that proliferative retinopathy induced by hypoxia (rather than diabetes) depends upon alphav (αV) integrin expression (E Chavakis et al., Diabetologia 45:262-267 (2002), which is hereby incorporated by reference in its entirety). It is proposed herein that PAR2 action on a specific integrin alphaVbeta-3 (αVβ3) and tissue factor/factor VIIa is permissive in the development of diabetic retinopathy.

PAR2 antagonists including monoclonal antibody, linear peptides such as HOOC-FSLLRY-NH₂ (SEQ ID NO: 1), COOH-LSIGRL-NH₂ (SEQ ID NO: 2), cyclic peptide analogs, non-peptide, and polymer conjugates can be used to inhibit angiogenesis to treat disorders associated with such excessive or pathological angiogenesis.

### EXAMPLES

Standard experimental methods were used to study the effects of PAR2 agonists and antagonists/inhibitors on endothelial cell tube assembly and new blood vessel formation.

### Example 1

### Materials

All reagents were chemical grade and purchased from Sigma Chemical Co. (St. Louis, Mo.) or through VWR Scientific (Bridgeport, N.J.). Cortisone acetate, bovine serum albumin (BSA), and gelatin solution (2% type B from bovine skin) were purchased from Sigma Chemical Co. (St. Louis, Mo.). M199 growth medium with Earl's salts, basic FGF, Insulin-Transferrin-Selenium-G Supplement (I-T-Se) 100×, Dulbecco's phosphate buffered salt solution (PBS) with and without Ca⁺² and Mg⁺², and 0.5 M EDTA were obtained from Gibco BRL (Grand Island, NY). Human umbilical vein endothelial cells (HUVEC), endothelial cell basal medium (serum-free, EBM), endothelial growth medium (EGM) (supplemented with growth factors, fetal calf serum), and 0.025% trypsin/0.01% EDTA solution were purchased from Clonetics Inc. (San Diego, Calif.). Human prostrate (TSU-Pr) tumor cells were obtained from American Type Culture Collection (Rockville, Md.). Matrigel® matrix and human collagen type III were purchased from Becton Dickinson (Bedford, Mass.). HEMA-3 fixative and staining solutions were purchased from Biochemical Sciences, Inc. (Swedesboro, N.J.). Fertilized chicken eggs were purchased from Charles River Laboratories, SPAFAS Avian Products & Services (North Franklin, Conn.).

PD 98059 from Calbiochem; Polyclonal anti-FGF2 and monoclonal anti-PAR2 were obtained from Santa Cruz Biotechnology and human recombinant FGF2 from Invitrogen.

### Example 2

### Inhibition of Endothelial Cell Tube Formation

Differentiation by endothelial cells was examined using a method developed by Grant et al. (Grant et al., In Vitro Cell Dev. Biol., 27A:327-336 (1991), which is hereby incorporated by reference in its entirety). Matrigel® matrix, phenol-red free (commercially available from Becton Dickinson, Bedford, Mass.) was thawed overnight at 4° C. Using cold pipette tips, 3.0 mg/well of Matrigel® matrix was placed in a cold twenty-four-multiwell plate (Falcon). Matrigel® matrix was allowed to polymerize during incubation at 37° C. for 30 minutes.

Human umbilical vein endothelial cells (HUVEC) were maintained at 37° C. with 5% CO₂ and 95% humidity in endothelial cell growth medium with 2% fetal bovine serum (EGM). The tube assay was performed in endothelial cell basal medium (EBM) supplemented with 0.5% bovine serum albumin (BSA) and 1:100 diluted Insulin-Transferrin-Selenium-G supplement (I-T-Se, 100×). HUVEC were trypsinized, centrifuged, and, subsequently, washed twice in phosphate buffered saline (PBS). After counting cells, cell density was adjusted to 35,000 cells/ml. A final concentration of 35,000 cells/ml/well was treated with human fibroblast growth factor basic (FGF2) at 100 ng/ml and PAR2 antibodies dissolved in EBM medium to a concentration of 0.015 μmol. Treated cells were incubated overnight at 37° C. with 5% CO₂ and 95% humidity to allow cell attachment. Subsequently, the medium was aspirated and cells were fixed and stained using a modified HEMA-3 stain kit. Digital images of micro-titer well sections were collected using a DKC5000 3-CCD color video camera system (Toshiba America, New York, N.Y.) and analyzed using Image-Pro Plus software (Media Cybernetics, Silver Spring, Md.). The area and major axis length of stained cells having a tubular morphology was measured on the Matrigel® matrix surface (Becton Dickinson, Bedford, Pa.).

The pro-angiogenesis effect of FGF2 and PAR2 activating agonists on endothelial cell tube formation are shown in Table 1.

TABLE 1
Effect of FGF2 and PAR2 on tube formation and its blockade
by PAR2 antagonist (PAR2 monoclonal antibody)

| | Mean Endothelial tube |
|---|---|
| Treatment | length (mm) ± SD |
| Control | 26 ± 3 |
| FGF2 (0.1 μg) | 64 ± 5** |
| PAR2 agonists¹ (0.25 μg) | 58 ± 4** |
| PAR2 agonist¹ + PAR2 antibody² | 29 ± 3 |

Data represent average branch points ± SD,
n = 3,
**P < 0.001
¹SEQ ID NO: 4
²From R&D Systems Inc. (Minneapolis, Minnesota)

### Example 3

### In Vitro 3D Sprout Angiogenesis of Human Microvascular Endothelial Cells

Culture of HDMEC on micro-carrier beads: 80% confluent HDMEC (Passage 5-10) were mixed with gelatin-coated Cytodex-3 beads with a ratio of 40 cells per bead. Suspend cells and beads (150-200 beads per well for 24-well plate) with 5 ml EBM+15% normal human serum, mix gently every one hour for first four hours, then leave the mixture culture in CO2 incubator overnight. The next morning, add 10 ml of fresh EBM+15% HS and culture for another three hours. Before experiments, check the culture of EC-beads. Add 500 μl of PBS in a well of 24-well plate, take 100 ul of the EC-bead culture solution, and add it to the PBS, observe and count the number of beads, calculate the concentration of EC-beads. The EC-beads are good for experiments for 48 hours.

Prepare fibrinogen solution (1 mg/ml) in EBM medium with or without angiogenesis factors or testing factors. For positive control, use 30 ng/ml VEGF+25 ng/ml FGF2. Wash EC-beads with EBM medium for two times and add EC-beads to fibrinogen solution. For each condition, do experiment in triplicates. Mix the EC-beads in fibrinogen solution gently and add 2.5 μl human thrombin (0.05 U/μl) in 1 ml fibrinogen solution, immediately transfer 300 ul to each well of 24-well plate. The fibrinogen solution will polymerize in 5-10 minutes, after 20 minutes, add EBM+20% normal human serum+10 μg/ml Aprotinin. Incubate the plate in CO₂ incubator. It takes about 24-48 hrs for HDMEC to invade fibrin gel and form tubes.

Human dermal micro-vascular endothelial cells (HDMVC) passage 11 were used. Images were taken at 4 and 10×, day 6. Cells were pretreated with FGF(2.5 ng)+VEGF(5 ng).

PAR2-mediated increase in vessel length is blocked by PAR2 antibody or the avb3 integrin antagonist, XT199.

TABLE 2
Effect of PAR1 versus PAR2 on angiogenesis in 3 D micro-vascular
endothelial cell sprouting (vessel tube length) assay

| HDMEC treatment | Vessel Length(mm) ± SEM |
|---|---|
| Control | 0.87 ± 0.06 |
| PAR-1(1 ug)² | 0.88 ± 0.08 |
| PAR-2(1 ug)¹ | 1.39 ± 0.28 |
| PAR-2(1 ug)¹ + XT199 (2 ug) | 0.72 ± 0.06 |
| PAR-2 (1 ug)¹ + PAR-2 antibody³ (1.5 ug) | 0.65 ± 0.09 |

¹SEQ ID NO: 5
²TFLLRN (SEQ ID NO: 6)
³From R&D Systems Inc. (Minneapolis, Minnesota)

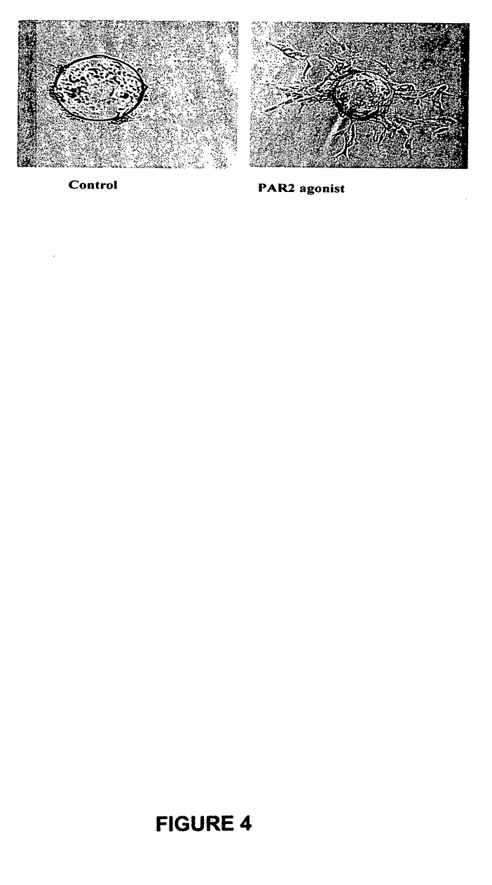

PAR2 agonists-induced endothelial sprouting (Table 2 and FIG. 4).

### Example 4

### Neovascularization in the Chick Chorioalolantoic Membrane (CAM) and Microscopic Analysis of CAM Sections

In vivo neovascularization was examined by the method previously described by Auerbach et al. (Auerbach et al., J. Dev. Biol., 41:391-394 (1974), which is hereby incorporated by reference in its entirety). Ten-day old embryos were purchased from Spafas, Inc. (Preston, Conn.) and were incubated at 37° C. with 55% relative humidity. In the dark with the help of a candling lamp, a small hole was punctured in the shell concealing the air sac with a hypodermic needle. A second hole was punctured in the shell on the broadside of the egg directly over an avascular portion of the embryonic membrane, as observed during candling. A false air sac was created beneath the second hole by the application of negative pressure to the first hole, which caused the chorioallantoic membrane (CAM) to separate from the shell. A window, approximately 1.0 cm², was cut in the shell over the dropped CAM with the use of a small crafts grinding wheel (Dremel, Division of Emerson Electric Company Racine, Wisconsin) which allowed direct access to the underlying CAM. Filter disks of #1 filter paper (Whatman International, United Kingdom) were soaked in 3 mg/ml cortisone acetate (Sigma, St. Louis, Mo.) in a solution of 95% ethanol and water and subsequently air dried under sterile conditions. FGF2 (Life Technologies, Gaithersburg, Md.) was used to grow vessels on the CAMs of 10 day old chick embryos. Sterile filter disks adsorbed with FGF2 dissolved in PBS at 1 μg/ml were placed on growing CAMs. Sterile filter disks adsorbed with FGF2 or PAR2 activating agonists were dissolved in PBS at 1 μg/ml were placed on growing CAM. At 24 h, test compounds or control vehicle was added directly to CAM topically.

CAM tissue directly beneath FGF2-saturated filter disk was resected from embryos treated 48 hours prior with test compound or control. Tissues were washed three times with PBS. Sections were placed in a 35-mm petri dish (Nalge Nunc, Rochester, N.Y.) and examined under a SV6 stereomicroscope (Karl Zeiss, Thornwood, N.Y.) at 50× magnification. Digital images of CAM sections adjacent to filters were collected using a 3-CCD color video camera system (Toshiba America, New York, N.Y.) and analyzed using Image-Pro Plus software (Media Cybernetics, Silver Spring, Md.).

CAM tissue directly beneath FGF2-saturated filter disk was resected from embryos treated 48 h prior with compound or control. Tissues were washed three times with PBS. Sections were placed in a 35-mm petri dish (Nalge Nunc, Rochester, N.Y.) and examined under a SV6 stereomicroscope (Karl Zeiss, Thornwood, N.Y.) at 50× magnification. Digital images of CAM sections adjacent to filters were collected using a 3-CCD color video camera system (Toshiba America, New York, N.Y.) and analyzed with the Image-Pro Plus software (Media Cybernetics, Silver Spring, Md.).

### Example 5

### Effect of PAR2 Agonist on Angiogenesis

TABLE 3
Pro-angiogenesis Effects of PAR1, PAR2 agonists versus FGF2
or VEGF in the CAM Model

| CAM Treatment | Branch Points ± SEM |
|---|---|
| PBS | 87 ± 8 |
| FGF2 (1 ug/ml) | 182 ± 11** |
| VEGF (2 ug/ml) | 168 ± 10** |
| PAR-1(3 ug)¹ | 101 ± 7 |
| PAR-2(0.3 ug)² | 197 ± 15** |

Data represent mean ± SD,
n = 8 per group.
¹TFLLRN (SEQ ID NO: 6)
²SEQ ID NO: 5

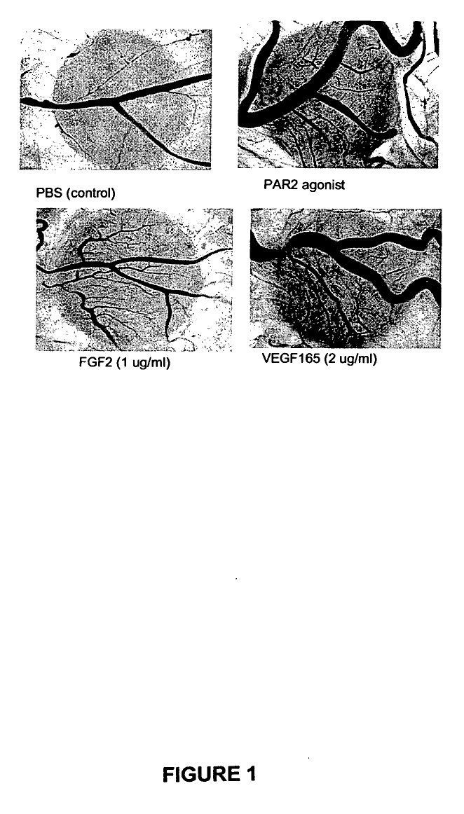

PAR2 agonist resulted in comparable pro-angiogenesis to that of FGF2 or VEGF (Table 3 and FIG. 1).

### Example 6

### PAR2 Antagonists and Angiogenesis

PAR2 activating agonists induced significant increase in angiogenesis index (2-3 fold increase above basal) in the CAM model. PAR2 activating agonists at 1.0-5.0 μg achieved maximal effect in producing 2-2.5 fold increase in angiogenesis index compared to 2-3 fold increase in angiogenesis index by 1 μg of FGF2 (Table 3 and FIG. 1). TF/Factor VIIA or PAR2 activating agonists produced comparable pro-angiogenesis effect to that observed with standard pro-angiogenic growth factors, such as FGF2, in the CAM model (Table 3). PAR antibody blocked FGF2, TF/Factor VIIA, and FGF2-induced angiogenesis in the CAM model of angiogenesis (Table 4). Table 4 contains the number of vessel branch points contained in a circular region equal to the area of a filter disk counted for each section.

TABLE 4
Effect of PAR2 antagonist on FGF2, PAR2 agonist or TF/Factor
VIIa-mediated stimulation of angiogenesis

| | Mean Number |
|---|---|
| Treatment | of Branch Points ± SD |
| Control | 76 ± 11 |
| FGF2 (1.0 μg) | 198 ± 14** |
| FGF2 + PAR2 antibody¹ | 78 ± 8 |
| PAR2 agonist² (5.0 μg) | 205 ± 8** |
| + PAR2 antibody¹ | 69 ± 9 |
| TF/factor VIIa | 188 ± 10** |
| TF/factor VIIa + PAR2 | 78 ± 6 |
| antibody¹ | |

DATA REPRESENT AVERAGE BRANCH POINTS ± SD,
N = 16,
**P < 0.001
¹From R&D Systems Inc. (Minneapolis, Minnesota)
²SEQ ID NO: 5

### Example 7

### Inhibition of Angiogenesis by PAR2 Antagonists and its Mediation via TF/factor VIIa, Integrin ανβ3, and MAP Kinase but not Thrombin

TABLE 5
Effect of XT199, anti-VIIa, PD98059, and Hirudin on PAR2
stimulated angiogenesis in the CAM Model

| | | Mean % |
|---|---|---|
| | Mean Branch | Inhibition ± |
| Treatment Group | points ± SEM | SEM |
| PBS | 86 ± 11 | |
| PAR-2¹ (0.3 ug/ml) | 176 ± 14 | |
| PAR-2¹ (0.3 ug/ml) + XT199 (10 ug) | 105 ± 8 | 86 ± 11 |
| PAR-2¹ (0.3 ug/ml) + anti-VII/VIIa (3 ug) | 110 ± 9 | 81 ± 9 |
| PAR-2¹ (0.3 ug/ml) + PD98059 (5 ug) | 102 ± 9 | 88 ± 12 |
| PAR-2¹ (0.3 ug/ml) + Hirudin (1 U) | 174 ± 9 | 3 ± 8 |
| PAR-2 (0.3 ug/ml) | 186 ± 17 | |
| PAR-2¹ (0.3 ug/ml) + PAR-2 mAb (3 ug) | 105 ± 9 | 83 ± 7.4 |
| PAR-2¹ (0.3 ug/ml) + anti-TF mAb (2 ug) | 96 ± 18 | 92 ± 15.0 |

Data represent mean ± SEM,
n = 8 per group
¹SEQ ID NO: 3

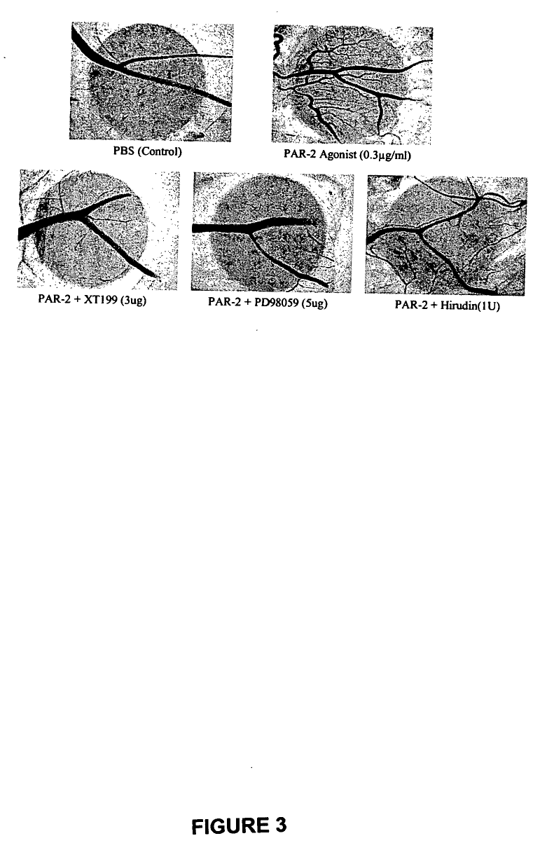

Effects of MAPK cascade inhibitors: PAR2-mediated stimulation of angiogenesis is blocked by MAP kinase inhibitor in the CAM model (Table 5 and FIG. 3).

PAR2-mediated stimulation of angiogenesis is blocked by ανβ3 antagonist (Table 5 and FIG. 3).

A major reason for heart failure following acute myocardial infarction is an inadequate response of new blood vessel formation, i.e., angiogenesis. PAR2 agonists are beneficial in heart failure and stimulate coronary angiogenesis. The methods of the present invention include, in part, delivering a single treatment of a PAR2 agonist at the time of infarction either by direct injection into the myocardium, or by simulation of coronary injection by intermittent aortic ligation to produce transient isovolumic contractions to achieve angiogenesis and/or ventricular remodeling.

### Example 8

### Wound Healing

Wound angiogenesis is an important part of the proliferative phase of healing. Healing of any skin wound other than the most superficial cannot occur without angiogenesis. Not only does any damaged vasculature need to be repaired, but the increased local cell activity necessary for healing requires an increased supply of nutrients from the bloodstream. Moreover, the endothelial cells which form the lining of the blood vessels are important in themselves as organizers and regulators of healing.

Thus, angiogenesis provides a new microcirculation to support the healing of wounds. The new blood vessels become clinically visible within the wound space by four days after injury. Vascular endothelial cells, fibroblasts, and smooth muscle cells all proliferate in coordination to support wound granulation. Simultaneously, re-epithelialization occurs to reestablish the epithelial cover. Epithelial cells from the wound margin or from deep hair follicles migrate across the wound and establish themselves over the granulation tissue and provisional matrix.

The role of topically applied PAR2 agonist or polymeric forms in wound healing, therefore, represents a novel strategy to accelerate wound healing in diabetics and in non-diabetics with impaired wound healing abilities. Topical administration can be in the form of attachment to a bandage. Additionally, nano-polymers and nano-particles can be used as a matrix for local delivery of PAR2 agonist and its analogs. This will aid in time controlled delivery into the cellular and tissue target.

Accordingly, another embodiment of the present invention features methods for treating wounds by promoting angiogenesis by administering to a subject in need thereof an amount of a polymeric form of PAR2 agonist, or an analog thereof, effective for promoting angiogenesis.

In Vitro 3D Wound Healing Assay of Human Dermal Fibroblast Cells:

Step 1: Prepare contracted collagen gels:

1) Coat 24-well plate with 350 μl 2% BSA at RT for 2 hr,
2) 80% confluent NHDF(normal human dermal fibroblast cells, Passage 5-9) are trypsinized and neutralized with growth medium, centrifuge and wash.

3) Prepare collagen-cell mixture, mix gently and always on ice:

| Stock solution | Final Concentration |
|---|---|
| 5 × DMEC | 1 × DMEM |
| 3 mg/ml vitrogen | 2 mg/ml |
| ddH₂O | optimal |
| NHDF | 2 × 10˜5 cells/ml |
| FBS | 1% |

4) Aspire 2% BSA from 24 well plate, add collagen-cell mixture 350 μl/well, and incubate the plate in 37° C. CO₂ incubator.
5) After 1 hr, add DMEM+5% FBS medium 0.5 ml/well, use a 10 μl tip Detach the collagen gel from the edge of each well, and then incubate for 2 days. The fibroblast cells will contract the collagen gel
   Step II: Prepare 3D fibrin wound clot and embed wounded collagen culture

1) Prepare fibrinogen solution (1 mg/ml) with or without testing regents. 350 μl fibrinogen solution for each well in eppendorf tube.

| Stock solution | Final Concentration |
|---|---|
| 5 × DMEC | 1 × DMEM |
| Fibrinogen | 1 mg/ml |
| ddH₂O | optimal |
| testing regent's | optimal concentration |
| FBS | 1% or 5% |

2) Cut each contracted collagen gel from middle with scissors. Wash the gel with PBS and transfer the gel to the center of each well of 24 well plate 3) Add 1.5 μl of human thrombin (0.25 U/μl) to each tube, mix well and then add the solution around the collagen gel, the solution will polymerize in 10 minute.
   After 20 minutes, add DMEM+1% (or 5%) FBS with or without testing agent, 450 μl/well and incubate the plate in 37° C. CO₂ incubator for up to 5 days. Take pictures on each day.
   In vivo Wound Healing in Diabetic Rats:

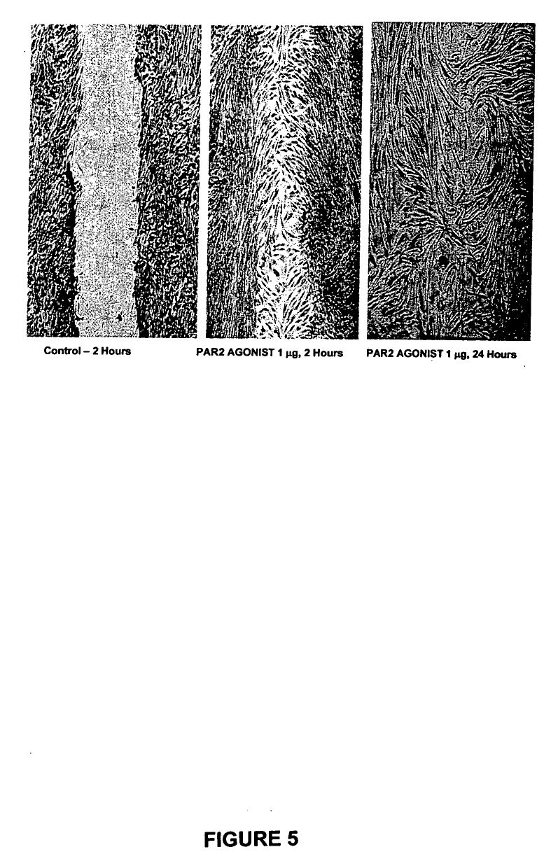

Using an acute incision wound model in diabetic rats, the effects of PAR2 agonists and its conjugated forms are tested. The rate of wound closure, breaking strength analyses and histology are performed periodically on days 3-21. See FIG. 5.

### Prophetic Example 9

### In vivo Angiogenesis in Matrigel FGF₂ or Cancer Cell Lines Implant in Mice

The murine matrigel model will be conducted according to previously described methods (Grant et al., In Vitro Cell Dev. Biol., 27A:327-336 (1991), which is hereby incorporated by reference in its entirety) and as implemented in Powell et al., J. Cellular Biochemistry 80:104-114 (2000), which is hereby incorporated by reference in its entirety. Briefly, growth factor free matrigel (Becton Dickinson, Bedford Mass,) will be thawed overnight at 4° C. and placed on ice. Aliquots of matrigel will be placed into cold polypropylene tubes and FGF2, PAR2 agonist or cancer cells (1×10⁶ cells) will be added to the matrigel. Matrigel with Saline, FGF2, PAR2 agonist or cancer cells will be subcutaneously injected into the ventral midline of the mice. At day 14, the mice will be sacrificed and the solidified gels will be resected and analyzed for presence of new vessels. The peptide of SEQ ID NOs: 3-5 will be injected subcutaneously at different doses. Control and experimental gel implants will be placed in a micro centrifuge tube containing 0.5 ml of cell lysis solution (Sigma, St. Louis, Mo.) and crushed with a pestle. Subsequently, the tubes will be allowed to incubate overnight at 4° C. and centrifuged at 1,500×g for 15 minutes on the following day. A 200 μl aliquot of cell lysate will be added to 1.3 ml of Drabkin's reagent solution (Sigma, St. Louis, Mo.) for each sample. The solution will be analyzed on a spectrophotometer at a 540 nm. The absorption of light is proportional to the amount of hemoglobin contained in the sample.

### Prophetic Example 10

### Tumor Growth and Metastasis—Chick Chorioallantoic Membrane (CAM) Model of Tumor Implant

The protocol is as previously described (Kim et al., Am. J Pathol 156:1345-1362 (2000), which is hereby incorporated by reference in its entirety). Briefly, 1×10⁷ tumor cells will be placed on the surface of each CAM (7 day old embryo) and incubated for one week. The resulting tumors will be excised and cut into 50 mg fragments. These fragments will be placed on additional 10 CAMs per group and treated topically the following day with 25 μl of compounds (the peptides of SEQ ID NOs: 3-5) dissolved in PBS. Seven days later, tumors will then be excised from the egg and tumor weights will be determined for each CAM.

The effects of PAR2 antagonists on tumor growth rate, tumor angiogenesis, and tumor metastasis of cancer cell lines can be determined.

### Prophetic Example 11

### Tumor Growth and Metastasis—Tumor Xenograft Model in Mice

The model is as described in Van Waes et al., Int. J Oncology 16:1189-1195 (2000) and Ali, et al., Cancer Research 60:7094-7098 (2000), each of which is incorporated herein by reference in its entirety). The anti-cancer efficacy for PAR2 antagonists at different doses and against different tumor types can be determined and compared.

### Prophetic Example 12

### Tumor Growth and Metastasis—Experimental Model of Metastasis

The model is as described in (Amirkhosravi et al., J. Thrombosis And Haemostasis, 1(9):1972-1976 (2003) and Amirkhosravi et al., Thrombosis And Haemostasis, 90(3):549-554 (2003), which are hereby incorporated by reference in their entirety). Briefly, B16 murine malignant melanoma cells (ATCC, Rockville, Md.) and other cancer lines will be cultured in RPMI 1640 (Invitrogen, Carlsbad, Calif.), supplemented with 10% fetal bovine serum, penicillin, and streptomycin (Sigma, St. Louis, Mo.). Cells will be cultured to 70% confluence and harvested with trypsin-EDTA (Sigma) and washed twice with phosphate buffered saline (PBS). Cells will be re-suspended in PBS at a concentration of either 2.0×10⁵ cells/ml for experimental metastasis. Animals: C57/BL6 mice (Harlan, Indianapolis, Ind.) weighing 18-21 grams will be used for this study. All procedures are in accordance with IACUC and institutional guidelines. The anti-cancer efficacy for PAR2 antagonists at different doses and against different tumor types can be determined and compared.

### Prophetic Example 13

### Retinal Neovascularization Model in Mice (Diabetic and Non-Diabetic

To assess the pharmacologic activity of a test article on retinal neovascularization, infant mice are exposed to a high oxygen environment for 7 days and allowed to recover, thereby stimulating the formation of new vessels on the retina. Test articles are evaluated to determine if retinal neovascularization is suppressed. The retinas are examined with hematoxylin-eosin staining and with at least one stain, which demonstrates neovascularization (usually a Selectin stain). Other stains (such as PCNA, PAS, GFAP, markers of angiogenesis, etc.) can be used. A summary of the model is below:
Animal Model
   Infant mice (P7) and their dams are placed in a hyper-oxygenated environment (70-80%) for 7 days.
   On P12, the mice are removed from the oxygenated environment and placed into a normal environment
   Mice are allowed to recover for 5-7 days.
   Mice are then sacrificed and the eyes collected.
   Eyes are either frozen or fixed as appropriate
   The eyes are stained with appropriate histochemical stains
   The eyes are stained with appropriate immunohistochemical stains
   Blood, serum, or other tissues can be collected
   Eyes, with special reference to microvascular alterations, are examined for any and all findings. Neovascular growth will be semi quantitatively scored.

Image analysis is also available.

A protocol disclosed in J de la Cruz et al., J. Pharmacol Exp Ther 280:454-459 (1997), which is hereby incorporated by reference in its entirety, is used for the administration of PAR2 antagonists (monoclonal antibody, cyclic peptide, or non-peptide) to rats that have streptozotocin (STZ)-induced experimental diabetes and diabetic retinopathy. The endpoint is the inhibition by PAR2 antagonist of the appearance of proliferative retinopathy (angiogenesis).

### Prophetic Example 14

### Polymer Compositions of PAR2 Agonist and Analogs

Polymer Conjugation through an Ester Linkage Using Polyvinyl Alcohol. In this preparation, commercially available polyvinyl alcohol (or related co-polymers) can be esterified by treatment with the acid chloride form of the peptides (SEQ ID NOs: 3-5). The hydrochloride salt is neutralized by the addition of triethylamine to afford triethylamine hydrochloride which can be washed away with water upon precipitation of the PAR2 agonist peptide ester polymer form for different analogs. The ester linkage to the polymer may undergo hydrolysis in vivo to release the active pro-angiogenesis PAR2 peptide agonist. Similarly, the linear peptide PAR2 antagonists (E and F) are conjugated using the above protocol.

### Prophetic Example 15

### Polymer Compositions of PAR2 Agonist and Analogs

For polymer conjugation through an anhydride linkage using acrylic acid ethylene co-polymer. This is similar to the polymer covalent conjugation or Prophetic Example 14; however, this time it is through an anhydride linkage that is derived from reaction of an acrylic acid co-polymer. This anhydride linkage is also susceptible to hydrolysis in vivo to release PAR2 agonist and analogs. Neutralization of the hydrochloric acid is accomplished by treatment with triethylamine and subsequent washing of the precipitated polyanhydride polymer with water removes the triethylamine hydrochloride byproduct. This reaction will lead to the formation of PAR2 agonist acrylic acid co-polymer+triethylamine. Upon in vivo hydrolysis, the PAR2 agonist will be released over time that can be controlled plus acrylic acid ethylene co-polymer. Similarly, the linear peptide PAR2 antagonists (E and F) are conjugated using the above protocol.

### Example 16

### Polymer Compositions of PAR2 Agonist and Analogs

For entrapment in a polylactic acid polymer, polylactic acid polyester polymers (PLA) undergo hydrolysis in vivo to the lactic acid monomer. This has been exploited as a vehicle for drug delivery systems in humans. Unlike the prior two covalent methods where the PAR2 agonist peptide is linked by a chemical bond to the polymer, this would be a non-covalent method that would encapsulate the PAR2 agonist into PLA polymer beads. This reaction will lead to the formation of PAR2 agonist containing PLA beads in water. Filteration and washing will result in the formation of PAR2 agonist containing PLA beads, which upon in vivo hydrolysis will lead to the generation of controlled levels of PAR2 agonist plus lactic acid. Similarly, the linear peptide PAR2 antagonists are conjugated using the above protocol.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A method of treating a subject for a condition mediated by a deficiency in angiogenesis, said method comprising:
administering to the subject a PAR2 agonist or activator under conditions effective to promote angiogenesis and treat the conditions mediated by a deficiency in angiogenesis.

2. The method of claim 1, wherein the PAR2 agonist is conjugated to a moiety selected from the group consisting of: polyvinyl alcohol, acrylic acid ethylene co-polymer, polyethylene glycol (PEG), and poly-lactic acid.

3. The method of claim 2, wherein the PAR2 agonist is conjugated to the moiety with a covalent or non-covalent bond.

4. The method of claim 3, wherein a covalent bond is utilized, said covalent bond being an ester linkage or an anhydride linkage.

5. The method of claim 1, wherein the PAR2 agonist is administered as a pharmaceutical formulation comprising a pharmaceutically acceptable carrier.

6. The method of claim 1, wherein the PAR2 agonist is encapsulated or incorporated in a microparticle, liposome, or polymer.

7. The method of claim 1, wherein said administering is carried out parenterally, orally, rectally, or topically.

8. The method of claim 1, wherein said condition mediated by deficiency in angiogenesis is selected from the group consisting of occlusive vascular disease, coronary disease, erectile dysfunction, myocardial infarction, ischemia, stroke, deep vein thrombosis, sickle cell diseases, pulmonary embolism, peripheral artery vascular disorders, and wounds.

9. The method of claim 1, wherein the PAR2 agonist can be used alone or in conjunction with other therapies for vascular disorders.

10. The method of claim 9, wherein the PAR2 agonist is co-administered with one or more compounds selected from the group consisting of a growth factor, a vasodilator, an anti-coagulant, and combinations thereof.

11. The method of claim 1, wherein the PAR2 agonist is encapsulated in a liposome or microparticle and lodges in capillary beds surrounding ischemic tissue.

12. The method of claim 1, wherein said administering is via a catheter.

13. The method of claim 12, wherein the PAR2 agonist is present in a polymeric system and said administering is within a blood vessel.

14. The method of claim 1, wherein the PAR2 agonist is co-administered with one or more compounds selected from the group consisting of a growth factor, a vasodilator, an anti-coagulant, and combinations thereof.

15. The method of claim 14, wherein a growth factor is co-administered with the PAR2 agonist, said growth factor being selected from the group consisting of transforming growth factor alpha (TGFα), transforming growth factor beta (TGFβ), basic fibroblast growth factor, vascular endothelial growth factor, epithelial growth factor, nerve growth factor, platelet-derived growth factor, and vascular permeability factor.

16. The method of claim 14, wherein a vasodilator is co-administered with the PAR2 agonist, said vasodilator being adenosine, adenosine derivatives, or combinations thereof.

17. The method of claim 14, wherein an anticoagulant or antithrombotic is co-administered with the PAR2 agonist, said anticoagulant or antithrombotic being heparin, heparin derivatives, Low Molecular Weight Heparin, anti-factor Xa, anti-thrombin, anti-tissue factor, anti-Factor VIIa, aspirin, clopidgrel, or combinations thereof.

18. The method of claim 14, wherein the PAR2 agonist is administered as a bolus injection prior to or after administering the growth factor, vasodilator, anti-coagulant, or combinations thereof.

19. The method of claim 1, wherein the PAR2 agonist is a linear or cyclic peptide, a peptidomimetic, or a polymer.

20. The method of claim 1, wherein said administering is carried out to promote angiogenesis along or around a medical device by coating the medical device with a PAR2 agonist prior to insertion into a patient.

21. The method of claim 20, wherein the PAR2 agonist is coated with a growth factor, a vasodilator, an anti-coagulant, or combinations thereof.

22. The method of claim 20, wherein said medical device is a stent, a catheter, a cannula, or an electrode.

23. The method of claim 1, wherein PAR2 agonist or activator is selected from the group consisting of linear peptides, cyclic peptides, non-peptide, trypsin, tryptase, coagulation Factor VIIa, Factor Xa, and their activators thereof.

24. A method of treating a subject for condition mediated by excessive or pathological angiogenesis, said method comprising:
administering to a subject a PAR2 antagonist or inhibitor under conditions effective to inhibit angiogenesis and treat the subject for the condition mediated by excessive or pathological angiogenesis.

25. The method according to claim 24, wherein an antagonist or inhibitor of Protease Activated Receptors 1, 3, 4, or combinations thereof is co-administered with the PAR2 antagonist or inhibitor.

26. The method of claim 23, wherein said condition mediated by angiogenesis is selected from the group consisting of: a primary or metastatic tumor and diabetic retinopathy.

27. The method of claim 24, wherein the PAR2 antagonist or inhibitor is a monoclonal antibody, cyclic peptide, non-peptides, or combinations thereof.

28. The method of claim 24, wherein said administering is carried out parenterally, orally, rectally, or topically.

29. The method of claim 24, wherein the PAR2 antagonist or inhibitor is co-administered with one or more other anti-angiogenesis therapeutics.

30. The method according to claim 29, wherein the anti-angiogenesis therapeutic is selected from the group consisting of integrin inhibitory compounds, angiostatin, endostatin, fibroblast growth factor inhibitors, fibroblast growth factor receptor inhibitors, vascular endothelial growth factor inhibitors, thrombospondin, platelet factor 4, interferon, interleukin 12, thalidomide, anti-tissue factor/anti-Factor VIIa, anti-VEGF, and combinations thereof.

31. The method according to claim 24, wherein the PAR2 antagonist or inhibitor is administered with a cytotoxic agent.

32. The method according to claim 31, wherein the cytotoxic agent is selected from the group consisting of nitrogen mustard, aziridine thiotepa, alkyl sulfonate, nitrosoureas, platinum complexes, alkylators, folate analogs, purine analogs, adenosine analogs, pyrimidine analogs, substituted urea, anti-tumor antibiotics, microtubule agents, asparaginase, and combinations thereof.

33. The method according to claim 24, wherein the subject is a cancer patient and the method is carried out to prevent and treat tumor growth, metastasis, and/or tumor angiogenesis.

34. The method according to claim 24, wherein said administering is carried out in conjunction with chemotherapy, radiotherapy, angiogenesis inhibitors, anti-inflammatory agents, and pre- and post-tumor surgery.

35. The method according to claim 24, wherein the subject has an angiogenesis-mediated disorder selected from the group consisting of tumors, cancer, ocular neovascular-disorders, inflammatory disorders, endometriosis, retrolental fibroplasia, rubeosis, capillary proliferation in atherosclerotic plaques, osteoporosis, Osler-Webber Syndrome, myocardial angiogenesis, plaque neovascularization, telangiectasia, hemophiliac joints, and wound granulation.

36. The method according to claim 24, wherein the subject has an inflammatory disorder as a result of rheumatoid arthritis or an inflammatory bowel disease.

37. The method according to claim 36, wherein the subject has an inflammatory bowel disease selected from the group consisting of ulcerative colitis and Crohn's diseases.

38. The method according to claim 24, wherein said administering is carried out in conjunction with co-administration of other therapeutics selected from the group consisting of non-steroid anti-inflammatory steroids, anti-TNF-α, and other cytokine and chemokine inhibitors.

39. The method according to claim 24, wherein said method is used to treat a solid tumor.

40. The method according to claim 24, wherein said method is used to treat retinal tissue or choridal tissue.

41. The method according to claim 40, wherein said administering is carried out to prevent and treat diabetic retinopathy, macular degeneration, or other ocular angiogenesis-mediated disorders.

42. The method according to claim 41, wherein said administering is carried out in conjunction with other therapies selected from the group consisting of other angiogenesis inhibitors, laser therapy, photodynamic therapy, and combinations thereof.

43. The method of claim 24, wherein PAR2 antagonist or inhibitor is selected from the group of PAR2 blocking linear peptides, cyclic peptides, peptidomimetics, non-peptides, anti-trypsin, anti-tryptase, direct or indirect inhibitors of coagulation Factor VIIa, and direct or indirect inhibitors of coagulation Factor Xa
